(19) United States
(12) Plant Patent Application Publication
(10) Pub. No.: US 2007/0039081 P1
(43) Pub. Date: Feb. 15, 2007

(54) PLUM TREE NAMED 'CONSTANZA'

(76) Inventor: Jose Domingo Godoy Huidobro, Santiago (CL)

Correspondence Address: WELLS ST. JOHN P.S., 601 W. FIRST AVENUE, SUITE 1300, SPOKANE, WA, 99201, US

(21) Appl. No.: 11/202,796

(22) Filed: Aug. 11, 2005

(51) Int. Cl. A01H 5/00

(52) U.S. Cl. PLT/184

(57) ABSTRACT

A new and distinct variety of plum tree “Prunus salicina” and which has been denominated as “Constanza,” and which further produces an attractively colored plum which is mature for harvesting and shipment about March 20^{th} to April 10^{th} under the ecological conditions prevailing in the Maipo Valley, near Santiago, Chile.

### BACKGROUND OF THE NEW VARIETY

The present invention relates to a new, novel, and distinct variety of plum tree “Prunus salicina” and which has been denominated varietally as “Constanza,” and more specifically to a plum tree which produces what are considered to be exceptionally high quality, black plums which are mature for harvesting, and shipping about March 20 to April 10 under the ecological conditions existing in the Maipo Valley of Santiago, Chile.

### ORIGIN AND ASEXUAL REPRODUCTION OF THE NEW VARIETY

The seedling “Constanza” was originated by me in 1990, and selected from among a population of seedlings which were derived from an open cross-pollination conducted between the “Larry Anne” plum tree, unpatented, which was the pollen parent; and “Angeleno” plum tree, U.S. Plant Pat. No. 2,747, and which was used as the seed parent. The resulting seeds from this cross-pollination were then planted in the spring of 1991. The seedlings which resulted from this group of seeds were then studied and observed during subsequent years. The present variety was selected from among the seedlings then growing in the orchard of the inventor which is located in Nos, San Bernardo, which is located in the Maipo valley of the Chilean Metropolitan Region. Once identified, the new variety was asexually reproduced and grafted onto Marianna 2624 rootstock (unpatented) for subsequent evaluation. The fruit and the trees produced from this subsequent asexual reproduction were then evaluated for their botanical, and post-harvest behavior for the next 5 years. It has subsequently been determined that these asexual reproductions run true to the original tree. The present tree is the subject of Chilean Plant Variety Rights Certificate 38/03, and which issued on Nov. 12, 2003.

### SUMMARY OF THE NEW VARIETY

The “Constanza” plum tree is characterized as to novelty, and is noteworthy by producing fruit which have what is considered to be a black skin coloration, and which ripen in the late season of maturity, and approximately 30 days after Angeleno plum tree, that is, approximately 20 March to about 10 April under the ecological conditions existing in the Maipo Valley of the Chilean Metropolitan Region. The fruit produced by the present variety is considered to be exceptionally sweet, and has a 16° brix in relative comparison to other varieties of plums which have a median of approximately 10° brix. In addition to the foregoing, the fruit produced by the “Constanza” plum tree is considered to be very large in comparison to the seed (mother) parent, that being the Angeleno plum tree. In relative comparison to its parents, the new variety produces low amounts of ethylene and has a significant blush. Moreover, the present variety has noteworthy storage and shipping qualities inasmuch as the fruit of the present variety has been kept in cold storage from 75 to 90 days with no deleterious effects noted. In comparison, the fruit produced by the “Larry Anne” plum tree typically can only be retained in storage for approximately 40 days; and the fruit produced by the “Angeleno” plum tree can typically only be kept in storage for about 60 days.

### BRIEF DESCRIPTION OF THE DRAWINGS

The colors in the enclosed photographs, which are provided, are as nearly true as is reasonably possible in color representations of this type. Due to chemical developments, processing and printing, the leaves and fruit depicted in these photographs may or may not be accurate when compared with the actual specimens. For this reason, future color reference should be made to the color plates (Royal Horticulture Society) and other descriptions provided for hereinafter.

FIG. 1 is a depiction of two whole mature fruit, and one fruit dissected substantially along the equatorial plane to expose the flesh and the pit thereof. Still further, several pits from the fruit of the present variety are shown in that view.

FIG. 2 shows both the dorsal and ventral coloration of typical leaves produced by the present variety.

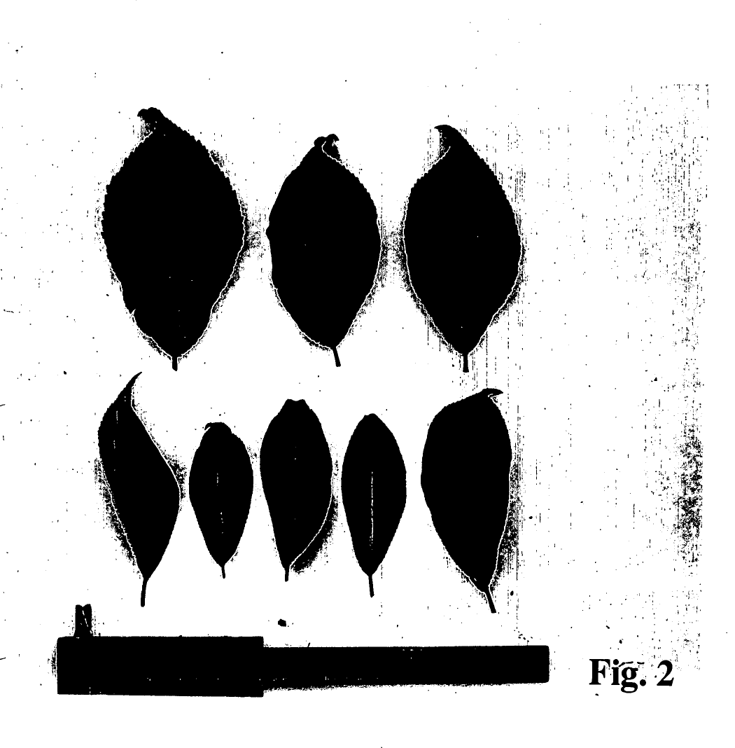

FIG. 3 depicts two whole fruit of the present variety taken from opposite positions.

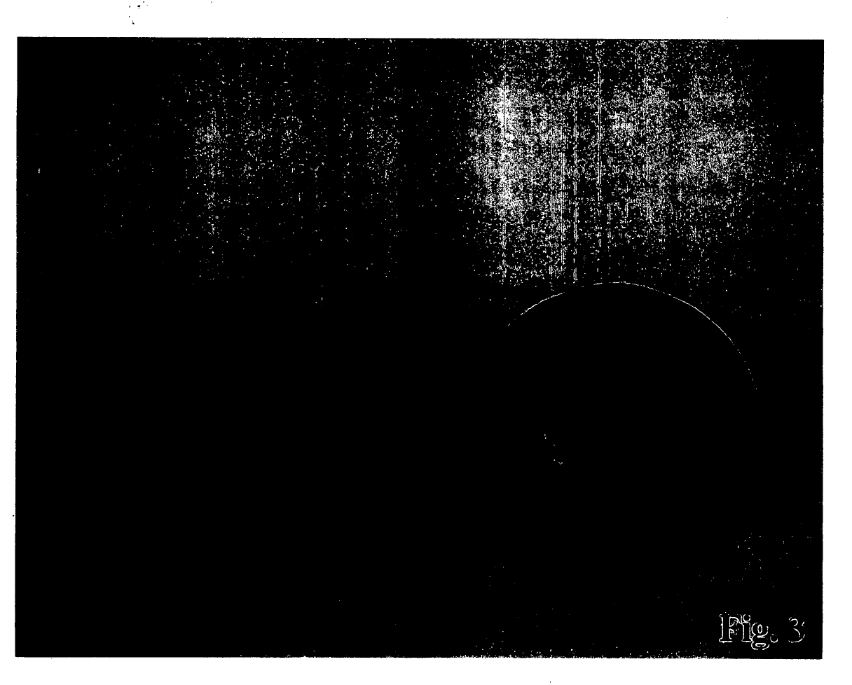

FIG. 4 shows characteristic twigs bearing typical leaves.

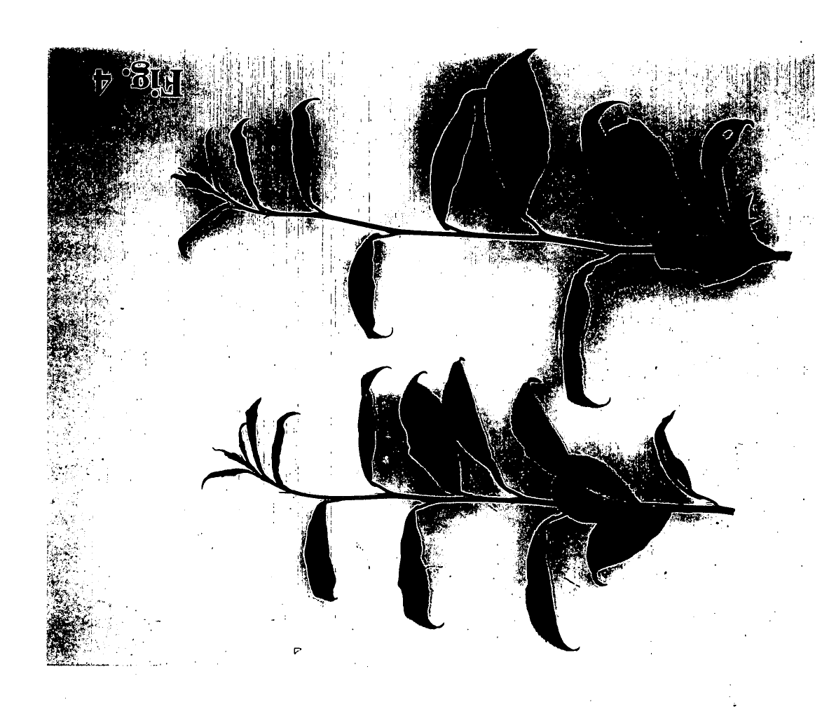

### DETAILED DESCRIPTION

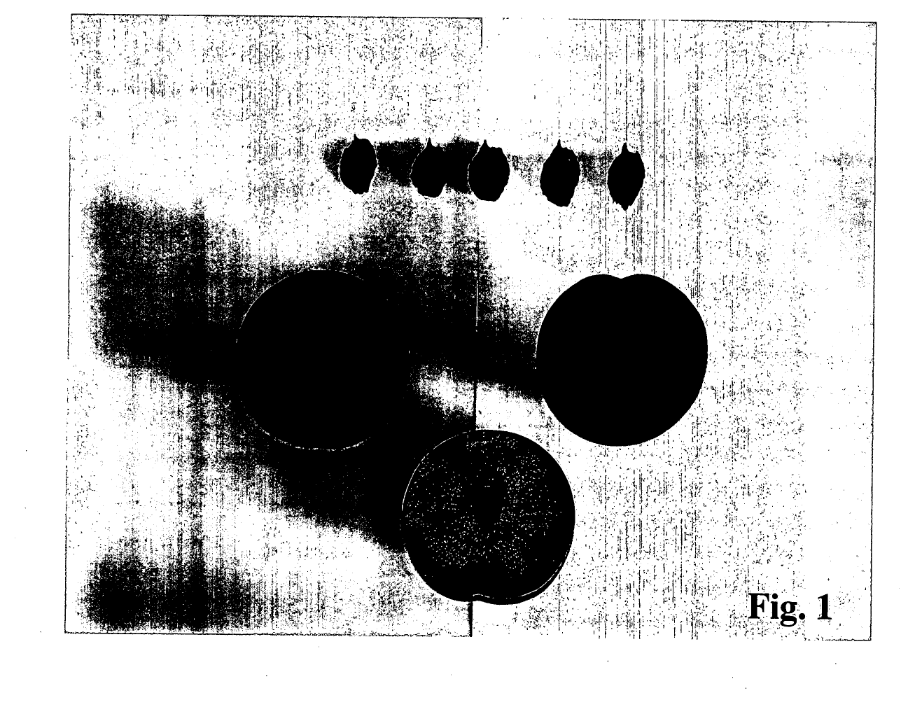

Referring more specifically to the pomological details of this new and distinct variety of plum tree, the following has been observed under the ecological conditions prevailing in the inventor's orchard which is located in the Maipo Valley of the Metropolitan Region of central Chile. All major color code designations are by reference to The R.H.S. Colour Chart (Edition 1995) and which is provided by The Royal Horticultural Society of Great Britain.
Tree:
   Size.—Generally — Medium, as compared to other plum trees varieties having a late date of maturity. The tree of the present variety was pruned to a height of approximately 320 cm. to about 340 cm. at commercial maturity.
   Productivity.—Considered very productive. The number of the fruit set varies with climatic conditions, and cultural practices employed during the blooming period and is typically considered not distinctive of the variety. However, trees 6 years and older have produced fruit which have an average weight of about 120 to 140 grams each.
   Vigor.—Vigorous in comparison with other common plum tree varieties. The present variety, when grafted onto Nemaguard rootstock (unpatented), has grown more than 300 cm during the first growing season. Still further, other plants grafted onto Marianna 2624 rootstock (unpatented) have grown from 200 to about 250 cm. during the first growing season. It has been observed that adult trees typically grow approximately 150 to 200 cm. in height during the growing season.
   Height.—Generally — Adult trees having an age of about 4-5 years generally attain a height of about 3.5 to 5 meters.
   Crown width.—Typically, adult trees of an age of 4-5 years attain a crown width of about 1.5 to about 2.5 meters.
   Figure.—Considered very erect and having a distinctive apical dominance.
Trunk:
   Size.—Considered average.
   Diameter.—Approximately 14 cm. when measured at a distance of approximately 30 cm. above the soil level. These measurements have been taken from trees that have been previously grafted on to Marianna 2624 rootstock. The measurements were taken at the end of the fourth growing season.
   Bark texture.—Considered moderately rough, but not distinctive of the variety.
   Bark color.—Grey-Green (RHS 197C)
   Lenticels.—Numerous flat, oval lenticels are present. The lenticels range in size from approximately 6 mm. to about 10 mm. in length; and about 1 mm. to about 3 mm. in width.
Branches:
   Size.—Considered to be average as compared to other common plum tree varieties.
   Diameter.—Four Year Old Trees — About 70 mm. to about 80 mm. when measured at a distance of about 110 cm. above the soil level.
   Diameter.—Current Season Shoots — About 5 mm. to about 6 mm.
   Internode length.—One Year Old Shoots — Approximately 8 to 12 mm.
   Internode length.—Current Season Shoots — Approximately 2 to 5 cm. when measured on trees which are four years old. This internode length is somewhat longer (3 cm. to 5 cm.) on one year old shoots.
   Surface texture.—Considered moderately rough.
   Crotch angle.—The present variety of plum tree has a very erect growing habit. Typically, primary branches have a crotch angle of about 70 to about 75 degrees when measured from the horizontal axis. This characteristic is not considered distinct of the variety, however.
   Lenticels.—Primary Branches — Numerous flat, oval lenticels are present. The lenticels range in size from about 6 mm. to about 10 mm. in length; and about 1 mm. to about 3 mm. in width.
   Lenticels.—One Year Old Shoots — Typically, lenticels are less evident on one year old shoots as compared to the primary branches. These lenticels are about 3 mm. in length; and about 1 mm. in width.
   Bark coloration.—Primary Branches — Grey-Green (RHS 197C).
   Color.—Current Season Shoots — Yellow-Green (RHS 144A). This color may vary by the end of the growing season and may be characterized as Grey-Orange (RHS 174A).
   Color.—One Year Old Shoots — Grey-Brown (RHS 199A).
Leaves:
   Size.—Considered average for the variety.
   Leaf width.—On average, approximately 32 mm. to about 36 mm.
   Leaf length.—On average, about 76 mm. to about 80 mm.
   Form.—Considered elliptical.
   Marginal form.—Considered crenate.
   Petiole.—Length — About 8 mm. to about 10 mm.
   Petiole.—Width — About 1 mm. to about 2 mm.
   Petiole color.—Yellow-Green (RHS 148C)
   Color.—Dorsal Surface — Considered a medium green (RHS 137B).
   Color.—Ventral Surface — Yellow-Green (RHS 147B).
   Mid-vein color.—Yellow-Green (RHS 148C).
   Glands.—Numbers — Considered few, and positioned on the base of the blade and on the petiole.
Flowers:
   Flower buds.—Size — Approximately 2 mm. to about 2.5 mm. in length; and about 1 mm. in width.
   Flower buds.—Surface Texture — Glabrous.
   Flower buds.—Form — Generally considered conic.
   Flower buds.—Color — Grey-Orange (RHS 176A).
   Flowers.—Size — Considered average for the variety. The flowers have an average diameter of approximately 17 mm. to about 20 mm.
   Aroma.—Considered non-aromatic.
   Flowers.—Form — The flowers have petals which overlap and touch.
   Stigma.—Position — Same level as compared with the anthers.
   Blooming period.—On average, about August 16^{th} to August 23^{rd} under typical Chilean environmental conditions.
   Petal.—Form — Circular.
   Flower color.—Considered White (RHS 155A).
   Petal size.—Considered average, and having a length of about 9 mm.; and a width of about 7 mm.
   Petals.—Marginal Form — Weakly undulate.
   Pollen production.—Present.
Fruit:
   Maturity when described.—Firm ripe for commercial harvesting and shipment approximately March 20^{th} to about April 10^{th} under typical Chilean environmental conditions.
   Size.—Considered large in comparison to other common varieties.
   Average cheek diameter.—Approximately 66 mm. to about 70 mm.
   Average axial diameter.—About 60 mm. to about 62 mm.
   Average fruit weight.—Typically about 120 grams to about 140 grams when the fruit has a pulp pressure of about 5 pounds to about 7 pounds. This average weight is dependent upon the ambient environmental conditions, and cultural practices, and is therefore not considered distinctive of this particular variety.
   Fruit form.—Considered rounded and somewhat flattened.
   Symmetry.—Considered slightly asymmetrical.
   Apex.—Shape — Considered flat.
   Stem cavity.—Size — Considered average.
   Stem cavity.—Width — Approximately 10 mm. to about 12 mm.
   Stem cavity.—Depth — Approximately 7 mm. to about 8 mm.
   Stem cavity.—Length — Approximately 12 mm. to about 14 mm.
   Flesh color.—Variable, but considered Yellow-Green (RHS 1C). This color may vary such that an increasingly yellow color appears in the region around the stone, and the Yellow-Green color appears near the skin as seen in FIG. 1.
   Flesh firmness.—Considered firm for the variety. The pulp pressure of about 5 pounds to about 7 pounds at commercial maturity appears to have contributed, at least in part, to a post-harvest storage lifetime of about 75 to 90 days.
   Juice production.—Considered medium juicy.
   Flesh flavor.—Considered excellent, and very sweet and having a medium level of acidity.
   Brix.—The present variety has a brix of typically about 16° at commercial maturity.
   Fruit skin.—Texture — The fruit skin displays a greater number of lenticels than the parent variety Larry Anne.
   Russet.—Present. The variety displays concentric rings of russet around the stem cavity thereof.
   Fruit blush.—Abundant. This appears to be an important factor in the post-harvest behavior of the present variety.
   Tendency of crack.—Not observed.
   Fruit skin color.—Dark Purple (RHS 187A). This is considered a black plum.
Stone:
   Type.—Considered semi-clingstone.
   Size.—Considered small for the variety.
   Stone length.—Approximately 24 mm. to about 25 mm.
   Stone width.—Approximately 15 mm. to about 16 mm.
   Stone thickness.—Approximately 9 mm. to about 10 mm.
   Stone form.—Generally considered to be elliptical and somewhat rounded in the side and basal views. It should be understood, however, that the stone size may vary depending upon the tree vigor, crop load and the prevailing growing conditions. Therefore the size of the stone is not particularly distinctive of the present variety.
   Stone symmetry.—Appears symmetrical when viewed ventrally and asymmetrical, when considered in profile.
   Stone.—Surface — Finely pitted.
   Dorsal groove.—Shape — Considered broken along the margin.
   Width of ventral zone.—Considered average for the variety.
   Pistil end.—Shape — Pointed.
   Shape of the stalk end of the stone.—Considered narrow.
   Tendency to split.—Not observed.
   Keeping quality.—Considered excellent. The present variety has been kept in cold storage conditions for 75 to 90 days with no deleterious effects noted.
   Use.—The present variety is a plum tree of the late season of maturity, and which has excellent consumer appeal, harvesting and shipping characteristics.
   Pollination.—The variety has been utilized with other pollinators such as the plum tree variety Larry Anne (unpatented), Wickson (unpatented), and Royal Diamond plum trees (U.S. Plant Pat. No. 6,756).

Although the new variety of plum tree possesses described characteristics when grown under the ecological conditions prevailing in the Maipo Valley of Chile, and which is located in the central zone of the Chilean Metropolitan Region, it should be understood that variations of the usual magnitude and characteristics incident to changes in growing conditions, fertilization, pruning, pest control and horticultural management are to be expected.

1. A new and distinct variety of plum tree substantially as illustrated and described, and which is characterized principally as to novelty by producing an attractively colored plum which is mature for harvesting and shipment approximately March 20^{th} to April 10^{th} under the ecological conditions in the Maipo Valley, near Santiago, Chile.
